(19) United States
(12) Patent Application Publication
(10) Pub. No.: US 2005/0177034 A1
(43) Pub. Date: Aug. 11, 2005

(54) Ear canal sensing device

(76) Inventor: Terry Beaumont, Cumberworth (GB)

Correspondence Address: MYERS BIGEL SIBLEY & SAJOVEC, PO BOX 37428, RALEIGH, NC, 27627, US

(21) Appl. No.: 10/506,340

(22) PCT Filed: Mar. 3, 2003
(86) PCT No.: PCT/GB03/00897

(30) Foreign Application Priority Data
Mar. 1, 2002 (GB) 0204887.4
Dec. 13, 2002 (GB) 0229073.2

(51) Int. Cl. A61B 5/00; H04R 25/00

(52) U.S. Cl. 600/323; 381/312; 600/549

(57) ABSTRACT

A sensing device is suitable for the non-invasive monitoring of physiological factors and is particularly suitable for location within the ear canal. The sensing device can be used to facilitate duplex audio communication.

The present invention relates to a sensing device suitable for the non-invasive monitoring of physiological factors, the device being particularly suitable for location within the ear canal. The invention also relates to provision for duplex audio communication for use with said sensing device.

Systems for the monitoring of critical physiological factors are known. U.S. Ser. No. 07/272,146 discloses the use of a nasal septum probe or oximeter. The application discloses the modified use of a nasal septum probe or oxisensor used with a conventional medical pulse oximeter. The nasal septum probe fits over a patient's nose bridge, or septum. The nasal septum oxisensor is modified to mount within the nose bridge portion of a conventional aircrew member face mask so that the blood oxygen saturation and pulse rate of the aircrew member can be monitored without any noticeable interference with, or extra effort by, the aircrew member.

A pulse oximeter calculates blood oxygen saturation from the different rates at which oxygenated haemoglobin and reduced haemoglobin absorb light of different wavelengths or frequencies. Typically, two wavelengths of light are used, one in the red portion of the spectrum and the other in the infra-red. Also typically, absorption of the infra-red wavelengths is much less sensitive to blood oxygen saturation levels than is absorption of the red wavelengths. The intensity of a particular infra-red wavelength remaining after passing through vascular tissue can serve as a constant against which to measure the intensity of a particular red wavelength remaining after passing through the same vascular tissue. Pulse rate is calculated from the timing of the relative rise and fall of the amount of light absorbed at each wavelength.

The pulse oximeter probe prior art has placed light emitting diodes (LEDs), and corresponding light sensors, over a variety of body appendages having sufficient vascular tissue. Such appendages include a finger, an ear pina, or ear lobe, the nasal septum as previously mentioned, and the scalp. The prior art refers to ear oximeters, but in most cases it refers to oximeters using probes, or bxisensors, that mount across the ear lobe.

U.S. Pat. No. 5,213,099 describes a sensing device comprising physiological sensing means in the form of a probe for measuring blood oxygen saturation level and a pulse monitor. The device comprises a means to locate the sensing means inside the ear canal. The device is specifically designed as a non-invasive, unobtrusive physiological monitor for a pilot or an aircrew member of high performance aircraft. Placement of the probe inside the relatively dark ear canal is greatly advantageous as it reduces sensitivity to error from external light sources and allows measurement of blood oxygen saturation at a location as near as possible to the blood supply to the brain of an air-crew member. This is due to the fact that the main artery that supplies the brain also supplies the timpanic membrane. The device is incorporated as part of a protective ear plug already issued to aircrew members.

Since the device of U.S. Pat. No. 5,213,099 effectively seals off the auditory meatus into which it is plugged, the user of the device suffers from various problems. These include an uncomfortable feeling of pressure in the ear canal, sweat in the ear, pollution by cerumen and loss of sound location when the auditory meatus is blocked off.

According to a first aspect of the present invention there is provided a sensing device comprising:
a) physiological sensing means; and
b) locating means to locate the sensing means inside an ear canal;
   characterised in that the locating means is provided with an aperture which, when the sensing device is fitted in the ear canal, allows motion of the air in and out of the ear canal.

Placement of the device of the present invention inside the ear canal or auditory meatus has the important advantages of reduced sensitivity to error from external light sources and measurement of important parameters such as blood oxygen saturation, heart rate, and body core temperature as near as possible to the blood supply to the brain of the wearer.

Preferably the locating means is substantially U-shaped and the aperture is defined by the trough between each arm of the U.

Preferably the locating means is made of pliable material and can be adapted to fit comfortably within the ear canal.

Preferably the locating means is provided with an adjusting means such that one device can be comfortably accommodated by a multiplicity of different users.

Most preferably the locating means is made of silicone or any other like material.

Preferably the locating means has a thermal conducting heat transfer tip.

Preferably the physiological sensing means comprises pulse oximetry optical transmitters and receiver.

Preferably the physiological sensing means also comprises a temperature sensor in contact with said heat transfer tip on the locating means.

Preferably the sensing device is provided with a securing means to secure the device to the ear of the user.

Preferably the securing means comprises an ear clip which partially or completely surrounds the ear.

The securing means may be designed to go around the top or bottom of the ear.

An alternative embodiment of the securing means is a custom made unit specifically to fit one ear of a particular user.

It will be appreciated that a means of audio communications between the user of the device and a person monitoring the physiological parameters will be desirable along with communication of physiological information and alarms for the wearer of the device.

Communication earpieces are known. U.S. Pat. No. 5,659,620 (Kuhlman) describes an ear microphone which is adapted to be disposed and retained in the outer ear region, i.e., outside the auditory meatus. The device is suitable for use with portable telephones or radios.

The disclosure of U.S. Pat. No. 5,659,620 teaches away from the use a device which is designed for arrangement within the auditory meatus.

Embodiments of the present invention seek to provide a physiological monitor.

Accordingly the sensing device of the present invention may further comprise an audio communications means comprising a speaker and a microphone.

Preferably a multi-core cable connects the sensing device to an interface unit for transmission by radio or cable to an external recording and monitoring means.

Preferably the speaker is located within the aperture of the U-shaped locating means.

Preferably the microphone contacts the front part of the outer ear. Preferably the microphone is a bone microphone and collects vibrations transmitted by the jawbone which passes near the front part of the outer ear. These vibrations are generated in the throat and vocal cords of the user upon talking.

Preferably the microphone aperture is pneumatically connected to a bubble of air. When the device is in use, the air bubble makes contact with the tragus against which it is placed and transmits vibrations in the air within the bubble to a microphone.

Preferably the communications means and the locating means are formed as separate components, each component suitably adapted for reversible attachment with said other component. Reversible attachment facilitates general maintenance and cleaning of the sensing device. In an alternative embodiment the communication means is integral with the locating means.

Applications of a present device of the present invention include but are not restricted to use within the emergency, medical and military services. The device also has applications within the industrial sector for the monitoring of workers in hazardous areas.

Due to the nature of potential applications of the present device mentioned above, it will be apparent to one of skill in the art that full duplex communication, as opposed to merely simplex communication, is clearly desirable. It is known that for an ear microphone and speaker to be used for full duplex, sufficient separation between input and output must be present in order to avoid self-resonance. Although U.S. Pat. No. 5,659,620 makes reference to the advantage of a full duplex communication the described embodiment does not enable such levels of communication.

According to a second aspect of the present invention there is provided a communications means comprising;
a) a speaker
b) a microphone
   characterised in that the speaker is located within a vibration absorbent material.

Preferably the speaker and/or the microphone are located within two layers of vibration absorbent material.

Preferably the surrounding enclosure for the speaker and microphone is a soft silicone sealant type material.

Preferably the location means is thermoplastic elastomer or thermoset silicone.

Preferably the location means vibration absorbing material has a sure hardness of 30 to 60%.

The absorbent materials serve to absorb vibrations emitted by the speaker and thus prevent transmittal of these vibrations to the microphone. Preferably the speaker is located partially withiin the inner auditory meatus thus increasing the distance between the speaker and the microphone. Accordingly the communication means of the present invention allows for full duplex communication.

The present invention is illustrated by the accompanying drawings wherein reference numerals 1 to 12 refer to the following earpiece parts:
1. earpiece body (lower)
2. venting aperture
3. infra-red & red transmitter
4. speaker
5. temperature sensor
6. microphone
7. U shaped locating means
8. optical sensors
9. microphone sound tube
10. microphone air bubble
11. ear piece body (upper)
12. multi-core cable

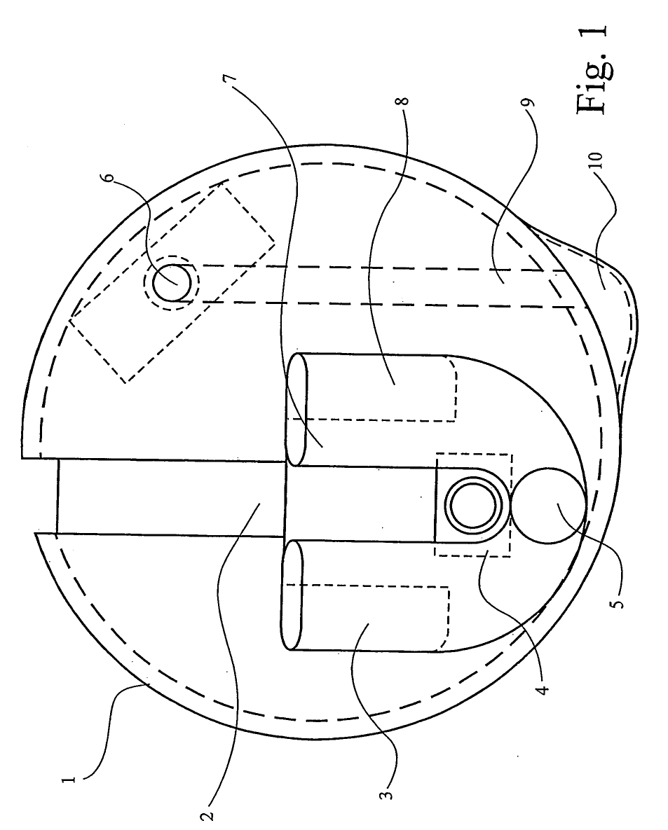

FIG. 1 illustrates in plan view, the detail of the lower earpiece body (1) and U shaped locating means (7).

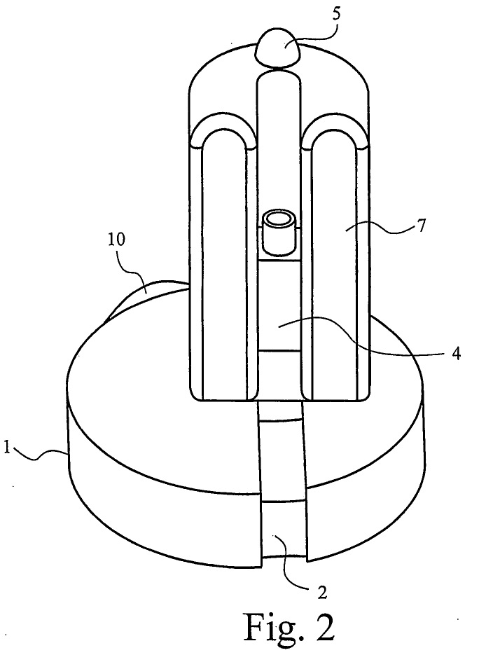

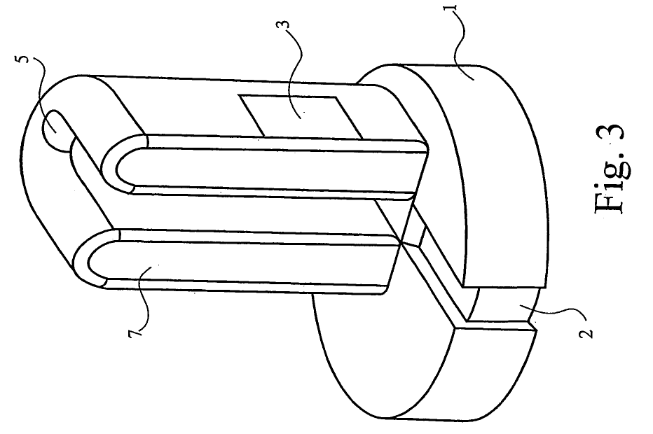

FIGS. 2 and 3 are perspective views and illustrate the aperture or groove provided by the U shaped locating means (7) and the location of the speaker (4) therewithin.

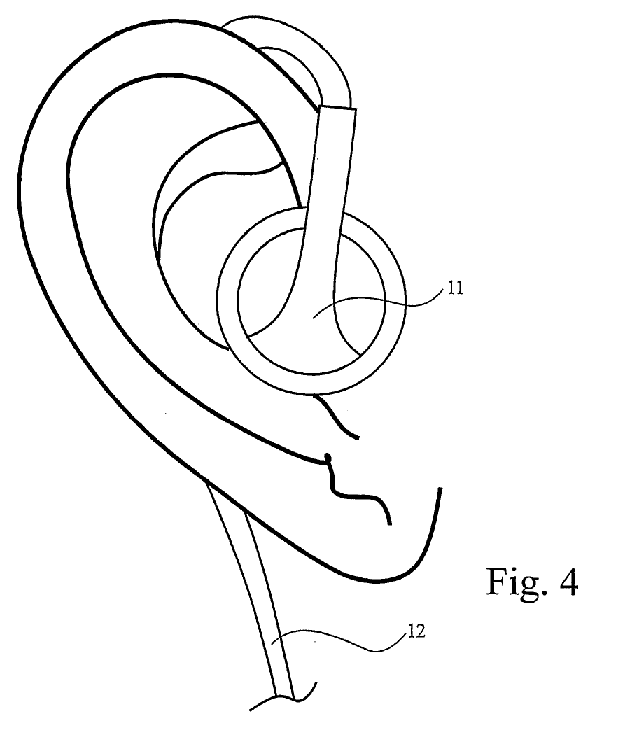

FIG. 4 shows the assembled sensing device located in the inner ear of a user.

1. A sensing device comprising:
physiological sensing means; and
locating means to locate the sensing means inside an ear canal;
wherein the locating means is provided with an aperture which, when the sensing device is fitted in the ear canal, allows motion of the air in and out of the ear canal.

2. A sensing device according to claim 1 wherein the locating means is substantially U-shaped and the aperture is defined by a trough between each arm of the U.

3. A sensing device according to claim 1 or wherein the locating means is made of pliable material and can be adapted to fit comfortably within the ear canal.

4. A sensing device according to claim 3 wherein the locating means is made of silicone.

5. A sensing device according to claim 1, wherein the locating means comprises adjusting means such that the device can be comfortably accommodated by a multiplicity of different users.

6. A sensing device according to claim 1, wherein the physiological sensing means comprises pulse oximetry optical transmitters and a receiver.

7. A sensing device according to claim 1, wherein the locating means has a thermal conducting heat transfer tip.

8. A sensing device according to claim 7 wherein the physiological sensing means further comprises a temperature sensor in contact with said heat transfer tip on the locating means.

9. A sensing device according to claim 1, wherein the sensing device comprises securing means to secure the device to the ear of a user.

10. A sensing device according to claim 9 wherein the securing means comprises an ear clip which partially or completely surrounds the ear.

11. A sensing device according to claim 9 wherein the securing means is designed to go around the top or bottom of the ear.

12. A sensing device according to claim 1, further comprising audio communication means wherein the audio communication means comprise a speaker and a microphone and the speaker and/or microphone is located within a vibration absorbent material.

13. A sensing device according to claim 12 wherein the absorbent material is a soft silicone sealant type material.

14. A sensing device according to claim 13 wherein the absorbent material is a thermoplastic elastomer or thermoset silicone.

15. A sensing device according to claim 12 wherein the absorbent material has a sure hardness of 30 to 60%.
